(19) United States
(12) Patent Application Publication
(10) Pub. No.: US 2004/0259240 A1
(43) Pub. Date: Dec. 23, 2004

(54) Method and apparatus for filtration of bioreactor recombinant proteins

(76) Inventor: Stephen J. Fadden, Harleysville, PA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/463,107

(22) Filed: Jun. 17, 2003

(51) Int. Cl. C12M 1/12; C12N 1/02

(52) U.S. Cl. 435/297.3; 435/261

(57) ABSTRACT

A substantially continuous filtration or separation system and related processes are disclosed. More specifically, a continuous perfusion cell culture system employing an external spin filtration system for the production of proteins is disclosed.

### FIELD OF THE INVENTION

The present invention relates to a substantially continuous filtration or separation system and related processes. More specifically, this invention includes a continuous perfusion cell culture system for the production of proteins. The invention provides, for example, a spin filtration system, external to a bioreactor, useful for, for example, increased production from a cell culture system, in which a filter can be replaced without terminating the cell culture batch.

### BACKGROUND OF THE INVENTION

Many pharmaceutically valuable proteins are produced using cell culture and recombinant cell culture techniques. In typical perfusion protein production systems, the recombinant protein-producing cells are maintained in a bioreactor, which also contains an internal filter that sieves the cells from the protein product-containing medium. These internal filters, however, often clog with cells or debris, causing technical delay or failure. More specifically, when an internal filter clogs, it can no longer separate product-containing medium from the cell culture. In that instance, the bioreactor process must be terminated, perhaps prematurely, sacrificing the cell culture, delaying production, and reducing product yield.

### SUMMARY OF THE INVENTION

The present invention discloses, for example, an advantageous bioreactor system with an external filtration apparatus that allows, for example, a cell culture to continue while a failed filter is replaced. The external filtration system may be optimized for commercial-scale production, and can prevent the premature termination of cell culture, thus allowing the completion of the bioreaction process and maximum product yield.

The objects of the present invention include, for example, providing methods, apparatus, and systems for the external filtration of cells from proteins produced from cell culture in a bioreactor. A particular embodiment of an external filtration apparatus and methods of use of the filtration apparatus include the steps of: providing cells that express a protein in a liquid cell culture medium, separating the culture medium that contains the expressed protein from the cell culture retentate using the rotatable filter, and removing the separated medium containing the expressed protein from the rotatable filter unit. In another embodiment of the present invention, any one of the foregoing steps may be paused. In yet another embodiment, separation of cell culture medium is accomplished by a rotating filter in which cell culture medium containing the expressed protein is harvested at a substantially slower rate than cell-containing medium is moved through the rotating filter apparatus.

In one embodiment of the present invention, separating the cell culture medium containing the expressed protein from the cells is also substantially continuous, as is the return of the cell retentate to the bioreactor. In another embodiment of the present invention, removing the separated culture medium containing the expressed protein is substantially continuous. In another embodiment of the present invention, removing the separated cell culture medium containing the expressed protein is facilitated by a device for raising, compressing or transferring fluids, such as a pump.

In an embodiment of the present invention, the harvested cell culture medium may be measured directly or indirectly, and this measurement may be used, for example, to allow for compensation of a volume deficiency by replenishing any volume deficiency with a sterile liquid culture medium. In one embodiment a load cell or scale may be used to measure weight. In another embodiment, flowmeters, or optical measurement means may be used to measure flow. In yet another embodiment, a pressure gauge may be used to indicate the pressure in the filtration reservoir.

In one embodiment of the present invention, an apparatus may be used to separate an expressed protein from cells in liquid cell culture. The apparatus may include a housing that is adapted to be external to a bioreactor, a rotatable filter inside of the housing, and one or more inlets into the housing. In another embodiment of the present invention, the inlet into the bioreactor is preferably adapted to receive a liquid cell culture medium that may contain an expressed protein. In yet another embodiment of the present invention, there is preferably a first and second outlet from the housing. In one embodiment of the present invention, the first outlet may be adapted to be in fluid communication with a bioreactor and may be adapted to return cell-containing retentate to a bioreactor from the housing. In another embodiment of the present invention, the second outlet may be in fluid communication with a harvest reservoir and may be used to transfer separated medium containing an expressed protein from the housing to the harvest reservoir.

In one embodiment of the present invention, the rotatable filter may include a basket. In another embodiment, the basket may be comprised of a mesh screen connected to a drum frame. In another embodiment of the present invention, the drum frame and mesh screen may be stainless steel. In yet another embodiment of the present invention, the drum frame and/or mesh screen may be adapted to be recyclable. In another embodiment of the present invention, the mesh screen is preferably annealed. In a further embodiment of the present invention, the drum frame may be adapted to be removable from the housing.

In an embodiment of the present invention, fluid flow may be substantially continuous. In another embodiment, fluid flow may be paused. In yet another embodiment of the present invention, one or more devices used to regulate the flow of fluid, such as a valve, for example, may be used to pause fluid flow. In another embodiment, the entire system may be closed to outside environments and may be sterile and/or cleanable. In yet another embodiment, the apparatus may be sterilizable by a steam supply source.

In one embodiment of the present invention, an indication system is used in conjunction with the apparatus and may consist, for example, of devices for measuring flow, weight, volume, and/or pressure.

Other objectives, features and advantages of the present invention will become apparent from the following detailed description. The detailed description and the specific examples, although indicating specific embodiments of the invention, are provided by way of illustration only. Accordingly, the present invention also includes those various changes and modifications within the spirit and scope of the invention that may become apparent to those skilled in the art from this detailed description.

### BRIEF DESCRIPTION OF THE DRAWINGS

FIG. 1 is a schematic diagram of an embodiment of the entire bioreactor and filtration apparatus system of the present invention.

FIG. 2 is a schematic diagram of an embodiment of the external filtration apparatus of the present invention.

### DETAILED DESCRIPTION OF THE INVENTION

It is understood that the present invention is not limited to the particular methodologies, protocols, media, cells, and proteins etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include plural reference unless the context clearly dictates otherwise. Thus, for example, a reference to “a protein” is a reference to one or more proteins and includes equivalents thereof known to those skilled in the art and so forth.

Unless defined otherwise, all technical and scientific terms used herein have the same meanings as commonly understood by one of ordinary skill in the art to which this invention belongs. Preferred methods, devices, and materials are described, although any methods and materials similar or equivalent to those described herein can be used in the practice or testing of the present invention. All references cited herein are incorporated by reference herein in their entirety.

The invention provides methods, apparatus, and systems for production and filtration of components such as, for example, proteins, specifically pharmaceutical or diagnostic proteins, expressed in cell culture. More specifically, the present invention relates to a novel external filtration apparatus useful in commercial-scale bioreaction and/or filtration. In one embodiment, the invention provides for the use of the external filtration apparatus in the initial protein isolation process, whereby the external separation of proteins from cells avoids premature termination of the bioreaction process that may be caused by failed filters. In this regard, the present invention provides an improvement in efficiency and flexibility over the prior art.

In a specific embodiment, the present invention relates to an external filtration apparatus and related methods of use, allowing for the production of proteins in cell culture, and preferably with an increase in yield and a reduction in production cost.

In a specific embodiment, the present invention relates to a fermentation and filtration system where cells are continuously cultured in a bioreactor or equivalent source and continuously flow through a retentate circuit. The retentate circuit is a closeable loop wherein the cell culture, suspended in a liquid media, flows out of the bioreactor, into the external rotatable filter apparatus in which cells are separated from protein-containing media, and this retentate cell culture is then returned to the bioreactor. Although the protein-containing media is separated from the cells, the technology described herein does not concentrate the protein; some protein may remain in the cell culture retentate that is returned to the bioreactor. The flow rate of the harvested protein-containing media out of the external filter may be substantially less than the flow rate of the retentate through the external filter unit, and this difference in flow rates may optimize performance of the external filter and the entire bioreactor circuit. Although the retentate circuit may generally be a closed loop system, it may be interrupted if the external spin filter clogs, allowing the filter to be separated and replaced, after which the circuit is again engaged.

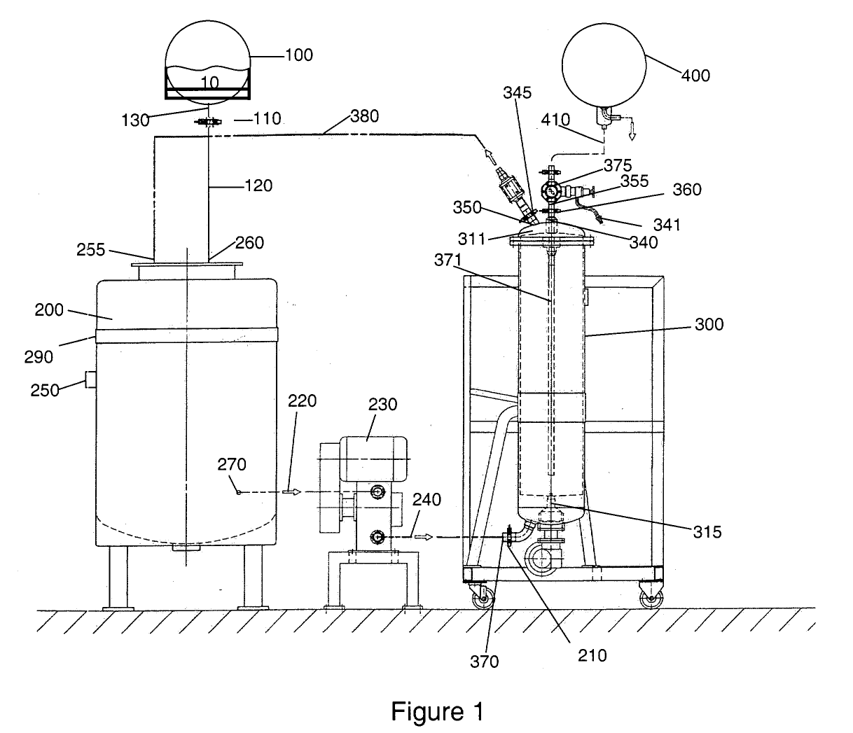

An embodiment of the bioreactor system of the present invention is depicted in FIG. 1. All components of this system may be and in this specific embodiment are designed to be sterilized and maintain sterility as required for Good Manufacturing Practices as outlined by the FDA. As shown in FIG. 1, a bioreactor system of the present invention may comprise a first reservoir 100, which may contain a fresh liquid medium 10. First reservoir 100 may be a large drum or similar container designed to contain fluid without oxidizing and maintain a sterile medium. First reservoir 100 may have an outlet 130, which is in fluid communication with a device used to regulate the flow of fluid, which may be a valve, such as, for example, a ball or butterfly valve, or any other device known in the art to be capable of regulating fluid flow that may be activated through manual or automatic motions. Valve 110 is in fluid communication with first reservoir 100 via outlet 130 and first conduit 120. First conduit 120 may be a tube, a pipe, or any other liquid transportation device, and may be made of any material suitable to sustain the weight and pressures of fluids and is of any suitable diameter. First conduit 120 may be made of a material designed to resist oxidation, such as stainless steel or plastic, and capable of containing sterile fluids without contamination. First conduit 120 is in fluid communication with valve 110 and bioreactor 200. In one embodiment, bioreactor 200 may be at a substantially lower elevation than first reservoir 100 to facilitate gravitational fluid flow from first reservoir 100 to bioreactor 200. In an alternative embodiment, for example, if filtration reservoir 300 is at the same or a higher elevation than bioreactor 200, a device for raising, compressing or transferring fluids, may be employed anywhere within the system to effect fluid flow. The device may include a pressurized system or a mechanical pump, such as a positive displacement pump, for example.

In an alternative embodiment, first reservoir 100, valve 110, and first conduit 120 may not be needed as bioreactor 200 may be filled by any external means known in the art.

Bioreactor 200 may be a large drum or similar container made of material, such as stainless steel, that may be cleaned and sterilized, should not oxidize, and may be large enough to be capable of commercial production, e.g., with an internal volume of about 500 liters to about 1000 liters. Bioreactor 200 may also have three inlets 250, 255, 260, and an outlet 270; however, bioreactor 200 may have any number of inlets or outlets. Inlet 250 provides an inlet for receiving cells; inlet 255 provides an inlet for receiving retentate cells; and inlet 260 receives fresh medium 10 from first reservoir 100. Bioreactor 200 may also contain an agitator 290, which may be a multi-bladed, or paddle mixer, or any other type of device used to mix or combine fluids that causes various components to mix together within bioreactor 200. In a specific embodiment, agitator 290 may be made of 316 L stainless steel or its equivalent, and may have a power capacity of about 2 hp. Second conduit 220 is preferably in fluid connection with pump 230. Pump 230 is in fluid connection with second conduit 220 and third conduit 240. Pump 230 may be a device for raising, compressing, or transferring fluids, such as for example, a positive displacement or peristaltic pump, made of 316 L stainless steel or its equivalent, and preferably has a power capacity of at least 1 hp. As stated earlier, a device for raising, compressing or transferring fluids, such as a pump may not be necessary to effect fluid flow; gravity or pressure, for example, may also be used. Third conduit 240 is in fluid connection with pump 230, valve 210, and a filtration reservoir 300. Valve 210 may be any type of valve, such as, for example, a ball or butterfly valve, or any other device known in the art to be capable of regulating fluid flow that may be activated through manual or automatic motions. Bioreactor 200 may also contain appropriate measuring and control devices for monitoring, adjusting and controlling volume, temperature, dissolved oxygen, pH, and cell density and any other relevant parameters.

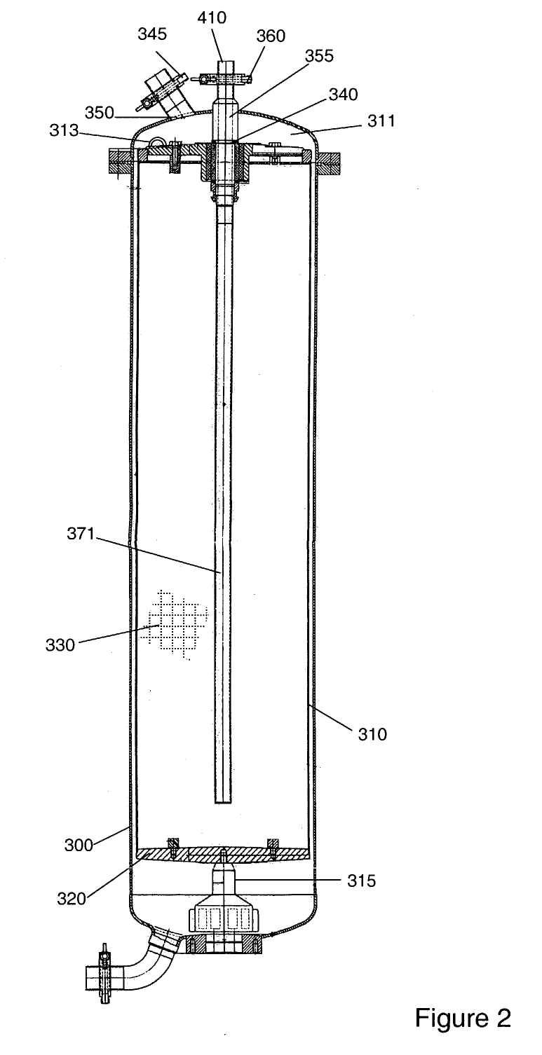

The external spin filter apparatus, shown in detail in FIG. 2, includes a filtration reservoir 300, which may be a large drum or similar rounded container, for example made of material that does not oxidize, such as 316 L stainless steel, and may be located at approximately the same height as bioreactor 200, such height affecting fluid flow by requiring a device for raising, compressing or transferring fluids, such as a pump to ensure substantially continuous flow. In an alternative embodiment, filtration reservoir 300 may be located at a lower height than bioreactor 200 to effect gravitational fluid flow, eliminating the need for a pump. In another alternative embodiment, filtration reservoir 300 may be located at any height and fluid flow may be accomplished by use of a pressurized system. In one embodiment, the height of the external filter housing may preferably be about 500 to 2000 centimeters and more preferably about 1314 centimeters, may have an outer diameter of about 100 to 400 centimeters and more preferably about 294 centimeters, an inner diameter of approximately 100 to 400 centimeters and preferably about 292 centimeters, and may be made of 316 L stainless steel or an equivalent material. The interior of filtration reservoir 300 preferably contains a basket 310 and fluid while in operation. Alternatively, the external filter housing may be of any dimensions capable of commercial-scale production.

Basket 310 may be a rotatable filter. In one embodiment, the height of basket 310 may preferably be 500 to 1200 centimeters and more preferably about 1134 centimeters in height, may have an outer diameter of approximately 100 to 300 centimeters, preferably 190 centimeters, an inner diameter of approximately 100 to 300 centimeters, preferably 170 centimeters, and may contain approximately 50 to 100 liters, preferably 80 liters. Basket 310 may be made of 316 L stainless steel, may have a frame 320, may be cylindrical or drum-shaped and may be adapted to have a hollow inner core. In an alternative embodiment, basket 310 may have other dimensions suitable for commercial-scale production and may be made of any material that is acceptable for contact with media and cell culture. Basket 310 may have mechanically sealed upper and lower ends, i.e., fluid may only enter the hollow inner core of basket 310 through mesh screen 330. The upper end of basket 310 may have a lifting eye 313 that also functions as the lid of the basket, is removable and contains other parts of a rotating seal and may also have a bearing rotatably connected to it for rotatable connection to harvest conduit 371 with, for example, a Teflon bearing block and o-rings; however basket 310 may be lifted by any devices known in the art and a rotatable connection may also be completed using other mechanical elements also known in the art. Frame 320 may have a mesh screen 330 welded to it. All welds may be polished in and out, and may also be passivated and electropolished. In an alternative embodiment, mesh screen 330 may be attached to frame 320 by any devices including fasteners, nails, bolts, adhesives, etc. that are suitable for contact with media and cells and are adequate for liquid submersion. Basket 310 is associated with a rotator 315, which allows rotation of basket 310 inside of filtration reservoir 300 and may be, for example, a magnetic drive, or an adjustable stirrer, each preferably capable of between about 60 and about 300 RPM.

Referring to FIG. 2, mesh screen 330 may be high quality steel such as, for example; 316 L steel, may have four mesh layers, and may be annealed and disposable. The screen type of mesh screen 330 may, for example, be Twill Dutch Weave, 11-18 microns, but may be of any appropriate caliber, dependent upon the desired cell retention rate, size, and type of cell. In a specific embodiment, mesh screen 330 may be annealed at a temperature of about 1000 degrees. This annealed mesh screen is commercially available from, e.g., GKD Gebr Kufferath GmbH & Co. (Dueren, Germany). Mesh screen 330 may also be made of any other suitable material capable of sieving cells and media and capable of maintaining its mechanical properties when exposed to fluids and rotational forces.

A harvest conduit 371 may be located in the hollow center of basket 310, may preferably be about 2.5 centimeters in diameter, may extend from lid 311 down through basket 310, ending approximately about 81 centimeters before the bottom of basket 310, and is the conduit through which the protein-containing medium is pumped out of filtration reservoir 300. In an alternative embodiment, the harvest conduit may be of any size and shape and may or may not contain any number of holes along its length inside reservoir 300. Harvest conduit 371 is preferably in fluid connection with harvest pump 375. Harvest pump 375 is preferably a positive displacement pump, may be made of Teflon, and may be capable of at least about ½ hp of power; however, any type of suitable pump or device for raising, compressing or transferring fluids may be used. In an alternative embodiment, harvest pump 375 may not be needed; fluid flow may be accomplished by gravity or by a pressurized system.

Referring to FIGS. 1 and 2, filtration reservoir 300 also may have a sealable lid 311, which may be sealed, for example, with two “o-ring” type seals. In an alternative embodiment, lid 311 may be sealed by any device known in the art. Sealable lid 311 has two outlets, 340 and 350. Outlet 340 may be substantially vertical, for example, but may be of any angle relative to filtration reservoir 300. Outlet 340 may also be in fluid connection with harvest conduit 371 and may also be associated with a valve 360. Valve 360 may be any type of valve, such as, for example, a ball or butterfly valve, or any other device known in the art to be capable of regulating fluid flow that may be activated through manual or automatic motions. Outlet 350 may be positioned at any angle relative to the lid 310 and is part of the retentate circuit and may have a device used to regulate the flow of fluid, such as a valve, for example. Valve 345 may be any type of valve, such as, for example, a ball or butterfly valve, or any other device known in the art to be capable of regulating fluid flow that may be activated through manual or automatic motions. The entire lid is connected to a steam supply source 341, or any other device for sterilization known in the art. At the bottom of filtration reservoir 300, there is an inlet 370, preferably below the bottom of basket 310, which is connected to third conduit 240 and may also be connected to a steam supply source for sterilization purposes. Filtration reservoir 300 is preferably connected to third conduit 240, fourth conduit 380, and a fifth conduit 355. Fourth conduit 380 is preferably in fluid connection with outlet 350 of filtration reservoir 300 and inlet 255 of bioreactor 200. Additionally, filtration reservoir 300 may be equipped with a pressure gauge and may also be adapted to connect with any number of conduits at any given elevation.

Fifth conduit 355 may be made of stainless steel or any other suitable or equivalent material, such as plastic, capable of sustaining fluid weight and flow without oxidizing, maintaining sterility, and may be of any suitable diameter. Fifth conduit 355 is preferably in fluid communication with filtration reservoir 300 via outlet 340 and harvest reservoir 400 and may also be in fluid communication with a valve 360. Harvest pump 375 is preferably in fluid connection with fifth conduit 355 and sixth conduit 410 and pumps protein-containing medium or any desired target substance from harvest conduit 371 through sixth conduit 410 to harvest reservoir 400. Valve 360 is preferably located on fifth conduit 355 in close proximity to filtration reservoir 300. Valve 360 may be any type of valve, such as, for example, a ball or butterfly valve, or any other device known in the art to be capable of regulating fluid flow that may be activated through manual or automatic motions. Sixth conduit 410 is preferably in fluid connection with harvest pump 371 and harvest reservoir 400.

Harvest reservoir 400 may be a large drum or similar container designed to contain fluid without oxidizing and may be located at a substantially higher elevation than filtration reservoir 300 and at approximately the same elevation as first reservoir 100. In an alternative embodiment, harvest reservoir 300 may be located at any height. Pumps, pressurization systems, and gravity may be used to generate fluid flow to harvest reservoir 300 at any height. Alternatively, a harvest reservoir may not be necessary for other embodiments.

All components in the bioreactor system that are in fluid contact with fresh media, cells, bioreactor contents, retentate cells and protein-containing media, may be made of stainless steel or some other type of metal or non-metal material, such as plastic, for example, that may be easily cleaned and sterilized, are able to contain fluids without contamination, resist iron oxidation, and are capable of handling fluids without leakage. Specific placement of components disclosed herein is not meant to be limiting; placement of valves, pumps, conduits, inlets, outlets, and reservoirs may be altered and will be apparent to those skilled in the art.

External Filtration Method of Operating Bioreactor

Referring to FIG. 1, before the operation of the bioreactor and external filtration method begins, the internal surfaces of all conduit means, reservoir means, valves, and any other assembly components are preferably sterilized. First reservoir 100 is preferably filled with fresh medium 10 and cells may then be placed inside bioreactor 200 through inlet 250 or in any other suitable manner known in the art.

In a method for fermentation and filtration, the method may begin when fresh medium 10 flows from first reservoir 100, which may be at a higher elevation to induce such flow, through outlet 130 and through first conduit 120. In an alternative embodiment, first reservoir 100 may be at any elevation and flow may be induced by gravity, pressure, or a device for raising, compressing or transferring fluids, such as a pump. Valve 110 may be used to control such flow through first conduit 120 and into bioreactor 200. Fresh medium 10 enters bioreactor 200 through inlet 260 at the top of bioreactor 200. Agitator 290 mixes fresh medium 10 with cells added through inlet 250. After proper fermentation time, the mixture then flows via gravity from bioreactor 200 through outlet 270 into second conduit 220. Alternatively, a pump or pressurized system may be used to induce such fluid flow. Pump 230 then pumps the mixture through conduit 240 against gravity into filtration reservoir 300. Valve 210 controls the flow of the mixture from pump 230 into filtration reservoir 300. Culture conditions, such as, for example, temperature, cell density, dissolved oxygen, pH, culture volume, and other relevant parameters may be monitored, controlled, and adjusted by appropriate means.

Referring to FIGS. 1 and 2, before receiving the cell and protein-containing medium from the bioreactor, the filtration reservoir 300 may be filled to a suitable level with a fresh, sterile medium via inlet 340 and valve 360. In an alternative embodiment, any inlet into filtration reservoir 300 may be used. The cell and medium mixture is preferably pumped into filtration reservoir 300 through inlet 370. The mixture may flow from the bottom or top of filtration reservoir 300 directly into the space between basket 310 and the inside wall of filtration reservoir 300. Basket 310 is preferably rotating and filtering the mixture such that protein-containing medium is pushed via pressure differential through mesh screen 330 to the interior of frame 320 while cells are drawn away from mesh 330 and remain in the retentate. Protein-containing medium may be drawn by harvest pump 375 into harvest conduit 371 at a significantly slower rate than the pump 230 moves cell culture medium through the retentate circuit. The harvested protein-containing medium then flows through outlet 340 into harvest reservoir 400. Meanwhile, cell culture retentate may remain outside basket 310 and move to the top of filtration reservoir 300 to outlet 350 into fourth conduit 380 back into bioreactor 200 via inlet 255, completing the retentate circuit.

Various indicators may monitor each portion of or the entire system. For example, the bioreactor may be monitored for volume, pH, temperature, dissolved oxygen content dissolved CO2 content, and cell density to ensure that bioreactor conditions are optimized. These indicators may be located at any appropriate locations. In one embodiment, a weight measurement means, such as a scale or a load cell, for example, is associated with the bioreactor that operates in conjunction with other measurements taken throughout the apparatus system and is preferably in electronic communication with a PC controller, or any suitable equivalent system that is capable of receiving and transmitting signals. In one embodiment, flow measurement means, such as flowmeters, are located in the inlets and outlets of filtration reservoir 300 and are also preferably in electronic communication with the PC Controller. In a specific embodiment, devices for measuring pH, temperature, weight, and flow may be automated and relevant measurements may be displayed on the PC Controller. The PC Controller is preferably electrically connected to the filtration apparatus and may calculate and/or control the synchronization of the weight and flow to ensure that the medium harvested via harvest conduit 371 and harvest pump 375 is preferably replaced by fresh medium to maintain equilibrium throughout the system, particularly in filtration reservoir 300. Optionally, the internal pressure of filtration reservoir 300 may be monitored. In another embodiment, the apparatus may be monitored and/or controlled manually.

The present invention is designed, for example, to avoid the problems associated with internal filter clogging, in which clogging may prematurely terminate a culture batch. The following description relates to exemplary processes used in conjunction with the external filtration apparatus of the present invention, if such filter clogs. A filter clog may be visually indicated, for example, by a pressure gauge that measures the pressure within filtration reservoir 300. A clog may also be indicated by inference, i.e., an increase in the weight of the bioreactor, which may indicate overfill. When a clog occurs, valves 210, 345, and 360 may be manually closed to isolate filtration reservoir 300 from any fluid flow in or out of it. In an alternative embodiment, the valves may be automated and may be closed if prompted by the system. The order in which the valves are closed may be varied in other embodiments. Next, in this embodiment, valve 210 closes and pumps 230 and harvest pump 375 are stopped. Valve 110 may also close to prevent flow of fresh medium 10 into bioreactor 200, but may also remain open to continue to feed bioreactor 200 during removal of the filtration basket or unit. Bioreactor 200 may continue culturing operations while the retentate circuit is interrupted.

Preferably, the entire filtration unit is removed from the room containing the bioreactor as soon as practical. In one embodiment, the filtration unit is preferably replaced immediately with an identical unit that has previously been cleaned and sterilized and is ready for immediate use. Alternatively, the initial cleaning steps, outlined below, are carried out such that an already prepared replacement basket may be inserted into the cleaned reservoir and the system sterilized and reinitiated as detailed below. The present invention is not meant to be limited to the specific cleaning and replacement process outlined below.

Referring to FIG. 2, the cleansing and sterilizing process may entail pumping deactivation fluids into inlet 350 or inlet 340 into filtration reservoir 300 and deactivating the fluid contents by means known in the art, such as chemical deactivation by NaOH. Next, filtration reservoir 300 may be drained and lid 311 removed. After lid 311 is removed, basket 310 is lifted and removed from filtration reservoir 300, by lifting eye 313 or any other appropriate means. Drum frame 320 may be recycled and mesh screen 330 may be disposed of and the unit washed and sterilized and a new mesh screen welded to drum frame 320. In an alternative embodiment, mesh screen 330 may be cleaned and reused.

Referring to FIG. 1, after the unit is either removed and replaced by a pre-cleaned unit or basket, or in an alternative embodiment, removed, cleaned, and reinserted into the system, steam supply sources such as 341 may be activated and steam sterilize all internal conduit means, valving means, inlets, or outlets. In an alternative embodiment, other devices known in the art may be used to sterilize those components exposed by the cleaning and removal process. The steam-sterilized components may then be cooled. After cooling, filtration reservoir may be filled with a sterile medium. The PC Controller may then be reset. Valving means, pumps, and filter rotation may be reactivated to allow fluid flow and filtration of the retentate circuit and harvest of protein-containing medium.

### EXAMPLES

Without further elaboration, it is believed that one skilled in the art, using the preceding description, can utilize the present invention to the fullest extent. The following examples are illustrative only, and not limiting of the remainder of the disclosure in any way whatsoever.

Myeloma cells capable of expressing a recombinant monoclonal antibody were removed from liquid nitrogen storage and warmed to ambient temperature in a warm water bath. Cells were washed and transferred to 3-L flasks containing 1.5-2 L media. A 150-L seed perfusion bioreactor was used to prepare culture for 1000-L production bioreactors. Inoculation viable cell density of about 2.0×10⁶ cells/ml was obtained in about four to about nine days.

The bioreactors were filled with either SFM_{—}8 or SFM_{—}10 medium. Agitation rates ranged from 10-45 rpm. Sparged carbon dioxide and air to the headspace allow pH control. Sparged air and/or oxygen were used to supply dissolved oxygen. Glucose, glutamine, lactate, and ammonia concentrations were also monitored. Bioreactor pressures range from about 1 to about 5 psig. Perfusion rates ranged from about 240 to about 870 L/day. Working volumes ranged from about 700 to about 1100 L. Biomass removal may be achieved using offline and inline pumps. Maximum viable cell densities range from 2.4×10⁶ viable cells/ml to 13.5×10⁶ viable cells/ml.

Several 1000-L production bioreactors were run with external spinfilter that could be changed-out upon fouling or mechanical failure, as described above. Spin filter screens were 10 μ-rated (165×1400 stainless steal weave), and either 19 cm or 25 cm in diameter, leaving a gap between the basket and the housing wall of 5 cm and 0.7 cm, respectively. External spinfilter speed may be set between about 100 to about 300 rpm, but optimal screen stability for the above screens was observed from about 135 to 150 rpm. Spinfilter duration ranged from 0.5 to 27 days, depending on cell density, with increased fouling when cell densities exceed 6×10⁶ viable cells/ml. At recirculation rates from about 280 to about 1125 L/hr, external spin filter speeds of about 100 to about 300 rpm and agitation rates of about 10 to about 45 rpm, cell viabilities were maintained at over eighty percent. Specific antibody production levels ranged from about 20 pg/vc/day to about 70 pg/vc/day. Maximum run length was about 60 days. Total product accumulation ranged from about 5 kg to about 12.2 kg for a single run. Parameters of these runs are summarized in Tables 1 and 2.

TABLE 1
Bioreactor Parameters, Specified Ranges, and Data.

| 1000 L | 1000 L | | | Peak Ab | Specific Ab |
|---|---|---|---|---|---|
| Bioreactor | Bioreactor | Peak Viable | | Titers | Production |
| Operation | Operation | Cell Density | Culture | Observed | Range |
| Parameter | Ranges | (×10⁶/mL) | Viability (%) | (mg/L) | (pg/vc/day) |
| Temperature | 35.5-37.5 | 13.5 | >80 | 622 | 20-84 |
| (C. °) | | | | | |
| pH | 6.90-7.50 | 13.5 | >80 | 622 | 20-84 |
| Dissolved O₂ | 25.0-55.0 | 13.5 | >80 | 622 | 20-84 |
| (%) | (air and O₂) | | | | |
| ESF Speed | 100-150 | 4.29-13.5 | >80 | 243-622 | 20-84 |
| (rpm) | | | | | |
| Agitation | 10-45 | 7.6-13.5 | >80 | 353-622 | 20-84 |
| (rpm) | | | | | |
| Pressure | 1.0-5.0 | 5.0-13.5 | >80 | 169-622 | 20-84 |
| (slpm) | | | | | |
| Recirculation | 563-1125 | 5.0-13.5 | >80 | 169-622 | 20-84 |
| Rate (L/hr) | | | | | |
| Working | 700-1190 | 7.1-13.5 | >80 | 431-622 | 20-84 |
| Volume (L) | | | | | |
| Medium Feed | 240-870 | 5.0-13.5 | >80 | 169-622 | 20-84 |
| Rate (L/day) | | | | | |

TABLE 2
Representative Operation Parameters.

| | | Viable | | | | |
|---|---|---|---|---|---|---|
| | | Cell | | | | |
| | Working | Density | | | ESF | Agitator |
| | Volume | Range | Recirculation | ESF | speed | Speed |
| Run | (L) | (×10⁶/mL) | Rate (L/hr) | Diameter | (rpm) | (rpm) |
| A | 730-1100 | 2.5-6.5 | 844 | 25 cm | 100- | 10 |
| | | | | | 135 | |
| B | 1100 | 0.8-3.8 | 844 | 25 cm | 100 | 10 |
| C | 1100 | 3.6-5.0 | 844 | 19 cm | 135 | 15 |
| D | 1100 | 5.0-5.8 | 844 | 19 cm | 135 | 15 |
| E | 1100 | 3.4-4.6 | 844 | 19 cm | 135 | 15 |
| F | 710 | 8.0 | 844 | 19 cm | 135 | 25 |
| G | 1100 | 6.7-7.4 | 844 | 19 cm | 135 | 35 |
| H | 1100 | 6.9-9.0 | 844 | 19 cm | 135 | 35 |
| I | 710 | 7.7-9.4 | 844 | 19 cm | 135 | 25 |

Several quality control tests were conducted. Bioreactor cell culture samples were removed from the bioreactors for mycoplasma and virus testing. All such tests were 10 negative. Overall charge heterogeneity, size, structure, and activity of the cA2 molecule produced in the bioreactor were measured by IEF (qualitative and quantitative), SDS-PAGE (reduced and non-reduced), GF-HPLC, mass spectral analysis, oligosaccharide profile and WEHI assay. These tests revealed no significant differences between samples and reference standards.

Various modifications of the described modes for carrying out the invention which are obvious to those skilled in molecular biology, mechanical engineering, or other related fields are intended to be within the scope of the present invention. The present invention is not meant to be limited by a particular protein, recombinant protein, or by the type of cell or media used in the bioreactor. Other embodiments of the invention, including general use for filtration and/or separation processes, will be apparent to those skilled in the art from consideration of the specification and the practice of the invention disclosed herein. It is intended that the specification and examples be considered as exemplary only, with a true scope and spirit of the invention being indicated by the following claims.

1. An apparatus capable of separating an expressed protein from retentate in liquid cell culture media produced in a bioreactor comprising:
a filtration reservoir, wherein said filtration reservoir is adapted to be in fluid communication with said bioreactor;
at least one inlet into said filtration reservoir, wherein said inlet is adapted to be in fluid communication with said bioreactor and wherein said inlet is adapted to receive said liquid cell culture from said bioreactor;
a rotatable filter located within said filtration reservoir, wherein said filter separates said media containing expressed protein from retentate in said filtration reservoir;
a first outlet from said filtration reservoir, wherein said outlet is adapted to be in fluid communication with said bioreactor and adapted to transfer said separated retentate to said bioreactor through said first outlet from said filtration reservoir; and
a second outlet from said filtration reservoir, wherein said outlet is in fluid communication with a harvest reservoir and adapted to transfer said separated media containing said expressed protein from said filtration reservoir to said harvest reservoir.

2. The apparatus of claim 1, wherein said rotatable filter further comprises a filtration basket.

3. The apparatus of claim 2, wherein said filtration basket comprises a drum frame.

4. The apparatus of claim 2, wherein said filtration basket comprises a mesh screen.

5. The apparatus of claim 4, wherein said mesh screen is annealed.

6. The apparatus of claim 4, wherein said mesh screen is adapted to be removable.

7. The apparatus of claim 3, wherein said drum frame is stainless steel.

8. The apparatus of claim 4, wherein said mesh screen is stainless steel.

9. The apparatus of claim 6, wherein said filtration basket comprises a mesh screen and a drum frame.

10. The apparatus of claim 6, wherein said filtration basket is adapted to be removable.

11. The apparatus of claim 7, wherein said drum frame is recyclable.

12. The apparatus of claim 8, wherein said mesh screen is disposable.

13. The apparatus of claim 1, wherein said at least one inlet is adapted for substantially continuous flow of said media from said bioreactor.

14. The apparatus of claim 1, further comprising at least one pump adapted for providing substantially continuous flow throughout said apparatus.

15. The apparatus of claim 1, wherein said first outlet is adapted for substantially continuous flow of media to said bioreactor.

16. The apparatus of claim 1, wherein said second outlet is adapted for substantially continuous flow to said harvest reservoir.

17. The apparatus of claim 1, further comprising a media-containing reservoir, wherein said reservoir is adapted to be in fluid connection with said bioreactor.

18. The apparatus of claim 1, wherein said apparatus is adapted to be sterilized.

19. The apparatus of claim 1, wherein said apparatus is sterilized by a steam supply source.

20. The apparatus of claim 1, wherein said apparatus is adapted to be cleaned.

21. The apparatus of claim 1, wherein the interior of said apparatus is capable of remaining isolated from the outside environment.

22. The apparatus of claim 1, further comprising one or more valves for discontinuing fluid flow through said at least one inlet into said filtration reservoir, wherein said one or more valves is in fluid connection with said at least one inlet and said filtration reservoir.

23. The apparatus of claim 1, further comprising one or more valves for discontinuing fluid flow through said first outlet to said bioreactor, wherein said one or more valves is in fluid connection with said first outlet and is adapted to be in fluid communication with said bioreactor.

24. The apparatus of claim 1, further comprising one or more valves for discontinuing fluid flow through said second outlet to said harvest reservoir, wherein said one or more valves is in fluid connection with said second outlet and said harvest reservoir.

25. The apparatus of claim 1, further comprising an indication system in electrical communication with a PC Controller comprising at least one member of the group consisting of a flowmeter for measuring flow of said separated media from said second outlet, a flowmeter for measuring flow of said liquid cell culture media containing said expressed protein fluid to said filtration reservoir, and one or more load cells for measuring the weight of said cell culture.

26. The apparatus of claim 25, further comprising a pressure gauge in connection with said filtration reservoir, wherein said pressure gauge is adapted to measure the internal pressure of said filtration reservoir.

27. The apparatus of claim 25, further comprising one or more valves for releasing fresh media from said media-containing reservoir to said bioreactor in fluid connection with said media-containing reservoir and said bioreactor, wherein said one or more valves for releasing fresh media from said media-containing reservoir is adapted to release said fresh media in an amount substantially equivalent to said fluid flow from said second outlet into said harvest reservoir.

28. The apparatus of claim 1, wherein said flow from said bioreactor to said filtration reservoir to said bioreactor comprises a retentate circuit.

29. A method for separating an expressed protein from cell retentate in liquid cell culture media comprising the steps of:
providing cells expressing said protein in liquid cell culture media;
separating said cell culture media into cell culture media containing said expressed protein and cell retentate using a rotatable filter;
returning said separated cell retentate from said rotatable filter to a bioreactor; and
removing the separated cell culture media containing said expressed protein from said rotatable filter.

30. The method of claim 29, wherein said removing step further comprises pumping a portion of separated cell culture media containing said expressed protein from said rotatable filter.

31. The method of claim 29, wherein said removing step further comprises storing the separated cell culture media containing said expressed protein from said filtration reservoir in a harvest reservoir.

32. The method of claim 29, wherein said separating step is substantially continuous.

33. The method of claim 29, wherein said returning step is conducted at a rate at least about ten times faster than said removing step.

34. The method of claim 29, wherein said removing step removes at least ten times slower than said transferring and returning steps.

35. The method of claim 29, wherein said returning step is substantially continuous.

36. The method of claim 29, further comprising the steps of
measuring said removed cell culture media containing said expressed protein; and
compensating for any volume deficiency caused by said removing step.

37. The method of claim 36, wherein said measuring step further comprises one or more of the following: measuring weight, measuring pressure, and measuring flow.

38. The method of claim 29, wherein said separating step may be paused.

39. The method of claim 29, wherein said returning step may be paused.

40. An apparatus capable of separating an expressed protein from retentate in liquid cell culture media comprising:
a filtration reservoir, wherein said filtration reservoir is external to a bioreactor and is adapted to be in fluid communication with said bioreactor;
a rotatable filter located within said filtration reservoir, wherein said filter has a removable filtration basket;
said filtration basket comprising an annealed mesh screen and a drum frame;
at least one inlet into said filtration reservoir, wherein said inlet is adapted to be in fluid communication with said bioreactor and wherein said inlet is adapted to receive said liquid cell culture from said bioreactor into said rotatable filter, and wherein said filter is adapted to separate a media containing expressed protein from said retentate in said filtration reservoir;
a first outlet from said filtration reservoir, wherein said outlet is adapted to be in fluid communication with said bioreactor and wherein said first outlet from said filtration reservoir is adapted to return said retentate to said bioreactor;
a second outlet from said filtration reservoir, wherein said outlet is in fluid communication with a harvest reservoir and wherein said separated media containing said expressed protein is transferred from said filtration reservoir to said harvest reservoir;
a pump adapted to be in fluid connection with said bioreactor and said filtration reservoir;
a second pump in fluid connection with said filtration reservoir and said harvest reservoir;
a steam supply source in fluid connection with said harvest reservoir, said filtration reservoir, and said bioreactor;
a first valve for discontinuing fluid flow through at least one inlet into said filtration reservoir in fluid communication with said filtration reservoir and adapted to be in fluid communication with said bioreactor;
a second valve for discontinuing fluid flow through said first outlet to said bioreactor, wherein said second valve is adapted to be in fluid communication with said bioreactor;
a third valve for discontinuing fluid flow through said second outlet to said harvest reservoir in fluid communication with said filtration reservoir and said harvest reservoir;
an indication system connected to a PC Controller comprising one or more of the following: a first flowmeter for measuring flow of said separated media from said second outlet, a second flowmeter for measuring flow of said liquid cell culture media containing said expressed protein fluid to said filtration reservoir, a pressure gage for measuring the pressure in said filtration reservoir, and a load cell for measuring the weight of said cell culture, wherein said indication system is adapted to be in electrical communication with said bioreactor, said harvest reservoir, said filtration reservoir, and said PC Controller;
a fourth valve adapted to provide fresh media in an amount that is substantially equivalent to said fluid flow from said second outlet into said harvest reservoir; and
a pump for removing said media containing expressed protein from said rotatable filter in fluid communication with said filtration reservoir and said harvest reservoir.

41. The apparatus of claim 40, wherein said mesh screen is adapted to be removable.

42. The apparatus of claim 40, wherein said drum frame is stainless steel.

43. The apparatus of claim 40, wherein said mesh screen is stainless steel.

44. The apparatus of claim 40, wherein said filtration basket is adapted to be removable.

45. The apparatus of claim 40, wherein said drum frame is recyclable.

46. The apparatus of claim 40, wherein said mesh screen is disposable.

47. The apparatus of claim 40, wherein said outflow from said second outlet to said harvest reservoir is substantially continuous.

48. The apparatus of claim 40, wherein said apparatus is adapted to be cleaned.

49. The apparatus of claim 40, wherein the interior of said apparatus is capable of remaining isolated from the outside environment.

50. The apparatus of claim 40, wherein said flow from said bioreactor to said filtration reservoir to said bioreactor comprises a retentate circuit.

51. An apparatus for separating an expressed protein from retentate in liquid cell culture media comprising:
a housing;
a filtration basket, wherein said filtration basket sits inside of said housing; and
a rotator rotatably connected to said basket.

52. The apparatus of claim 51, wherein said filter is adapted to be external to a bioreactor.

53. The apparatus of claim 51, wherein said filtration basket rotates substantially continuously.

54. The apparatus of claim 51, wherein said filtration basket is adapted to be replaceable.

55. The apparatus of claim 51, wherein said filter is adapted to be separable from a fermentation system.

56. The apparatus of claim 51, wherein said filter is adapted for commercial-scale production.

57. The apparatus of claim 51, wherein said filtration basket further comprises a drum and a mesh screen.

58. The apparatus of claim 57, wherein said mesh screen is annealed.

59. The apparatus of claim 57, wherein said mesh screen is stainless steel.

60. The apparatus of claim 57, wherein said frame is stainless steel.

61. The apparatus of claim 57, wherein said drum frame is adapted to be recyclable.

62. The apparatus of claim 57, wherein said mesh screen is adapted to be disposable.
